(19) United States
(12) Patent Application Publication
(10) Pub. No.: US 2026/0094381 A1
(43) Pub. Date: Apr. 2, 2026

(54) METHODS OF GENERATING IMAGE DATA FOR THREE-DIMENSIONAL TOPOGRAPHICAL VOLUMES, INCLUDING DICOM-COMPLIANT IMAGE DATA FOR SURGICAL NAVIGATION, AND ASSOCIATED SYSTEMS, DEVICES, AND METHODS

(71) Applicant: MONTEFIORE MEDICAL CENTER, Bronx, NY (US)

(72) Inventors: Oren Tepper, Bronx, NY (US); Donald Salisbury, Bronx, NY (US); Alex Gordon, Bronx, NY (US)

(21) Appl. No.: 19/113,335

(22) PCT Filed: Sep. 22, 2023
(86) PCT No.: PCT/US2023/074848

§ 371 Date: Mar. 19, 2025

Related U.S. Application Data
(60) Provisional application No. 63/376,850, filed on Sep. 23, 2022.

(51) Int. Cl. G06T 19/00; A61B 34/10; A61B 34/20; G06T 5/70; G06T 7/60; G06T 7/70; G06T 15/08; G06T 17/20; G16H 30/20

(52) U.S. Cl. CPC G06T 19/003; A61B 34/10; A61B 34/20; G06T 5/70; G06T 7/60; G06T 7/70; G06T 15/08; G06T 17/20; G16H 30/20; A61B 2034/105; A61B 2034/2065; G06T 2207/30004; G06T 2210/41

(57) ABSTRACT

Methods of generating image data for three-dimensional topographical volumes, and associated systems, devices, and methods are disclosed herein. In one embodiment, a method of generating a volumetric dataset for surgical navigation includes obtaining a three-dimensional graphical volume of a patient representing one or more surface contours of patient anatomy. The method can further include voxelating the three-dimensional topographical volume to generate a three-dimensional voxelated volume, and volume rendering the three-dimensional voxelated volume to a sequence of two-dimensional cross-sectional images that can each include a two-dimensional slice of the three-dimensional voxelated volume. The method can include conforming the sequence to an imaging standard (e.g., the DICOM Standard). In some embodiments, a difference between actual patient anatomy and desired patient anatomy at a location a probe contacts the patient can be displayed by a surgical navigation system once a three-dimensional volume reconstructed from the conformed sequence is registered to the patient.

### CROSS-REFERENCE TO RELATED APPLICATION(S)

The present application claims the benefit of U.S. Provisional Ser. No. 63/376,850, filed Sep. 23, 2022, which is incorporated by reference herein in its entirety.

### TECHNICAL FIELD

The present disclosure is directed to methods of generating image data for three-dimensional (3D) topographical volumes, and associated systems, devices, and methods. For example, several embodiments of the present technology are directed to generating image data (a) that is compliant with the Digital Imaging and Communications in Medicine (DICOM) Standard, (b) that is generated based at least in part on 3D topographical images of a patient, and/or (c) that can be used to reconstruct a 3D volume of the patient for use in surgical navigation systems during surgery on the patient's soft tissue or topographical anatomy.

### BACKGROUND

Computer-assisted surgery is a medical concept that often involves generating an accurate model of a patient, registering the model to the patient, and using the registered model for guiding or performing surgical interventions. The model is typically generated by capturing CT and/or MRI images of the patient, and then processing the images to generate a virtual model of the patient. The virtual model can be manipulated to provide views of the patient from a variety of angles and at various depths within the model. Using the model, a surgeon can plan and simulate a surgical intervention before surgery is actually performed on the patient. At the time of surgery, a surgical navigation system can be used to register the virtual model to the patient, display the model, track medical instruments used by the surgeon, and represent a position of the medical instruments at corresponding locations within the display of the model. The display of the model and the locations of the medical instruments can be used as a guide for the surgeon to perform the surgical intervention. Computer-assisted surgery is especially helpful to navigate medical instruments throughout patient anatomy when the medical instruments are inserted into the patient and obscured from the surgeon's view.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure. The drawings should not be taken to limit the disclosure to the specific embodiments depicted, but are for explanation and understanding only.

FIG. 1 is a partially schematic representation of a modeling and navigation system configured in accordance with various embodiments of the present technology.

FIG. 2 is a flow diagram illustrating a method of generating and using image data, such as DICOM-compliant image data for surgical navigation, in accordance with various embodiments of the present technology.

FIG. 3 is a display of a composite 3D topographical model of a patient having (i) a 3D topographical mesh representing actual patient anatomy and (ii) a 3D topographical mesh representing desired patient anatomy, the display and the composite 3D topographical model each configured in accordance with various embodiments of the present technology.

FIG. 4A is a display of a 3D topographical mesh of patient anatomy, the display and the 3D topographical mesh each configured in accordance with various embodiments of the present technology.

FIG. 4B is a display of a 3D volume generated by voxelating the 3D topographical mesh of FIG. 4A, the display and the 3D voxelated volume each configured in accordance with various embodiments of the present technology.

FIG. 4C is a display of a 3D voxelated volume generated by smoothing the 3D voxelated volume of FIG. 4B, the display and the smoothed 3D voxelated volume each configured in accordance with various embodiments of the present technology.

FIG. 5 is a display of a two-dimensional (2D) cross-sectional image generated during volume rendering of a 3D voxelated volume, the display and the 2D cross-sectional image each configured in accordance with various embodiments of the present technology.

FIG. 6 is a display of a pre-processed 2D cross-sectional image generated during simultaneous volume rendering of two 3D voxelated volumes, the display and the pre-processed 2D cross-sectional image each configured accordance with various embodiments of the present technology.

FIG. 7 is a display of DICOM-compliant image data using DICOM-compliant medical imaging software; the display, the image data, and the medical imaging software each configured in accordance with various embodiments of the present technology.

FIG. 8 is a display of (i) a first 2D image slice representing desired patient anatomy and overlayed onto (ii) a second 2D image slice representing actual patient anatomy; the display, the first 2D image slice, and the second 2D image slice each configured in accordance with various embodiments of the present technology.

FIG. 9 is a partially schematic perspective view of a physical instrument configured in accordance with various embodiments of the present technology and contacting a patient's forehead in accordance with various embodiments of the present technology.

### DETAILED DESCRIPTION

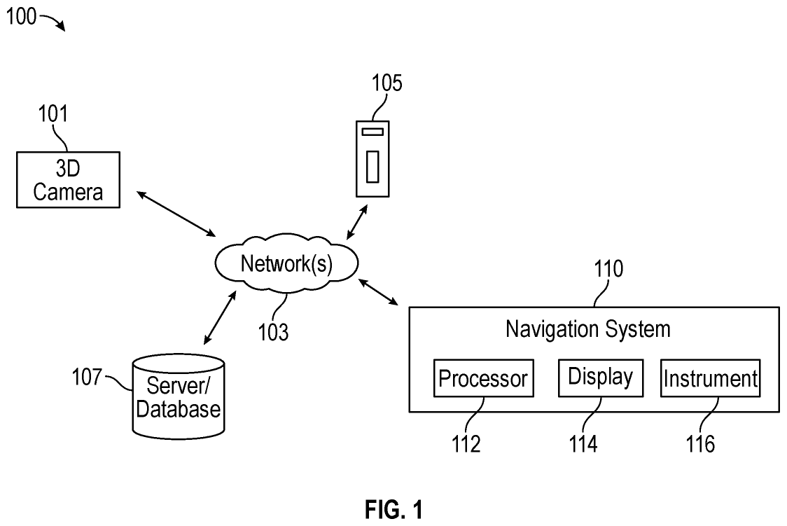

The present disclosure is directed to methods of generating image data for three-dimensional volumes, and associated systems, devices, and methods. For example, in the illustrated embodiments below, the present technology is primarily described in the context of generating DICOM-compliant image data from three-dimensional topographical images of a patient to construct 3D topographical volumes of the patient that can be used in surgical navigation systems to conduct surgery on the patient. Image data generated in accordance with various embodiments of the present technology, however, can be of objects other than patients, can be generated in compliance with another imaging standard, can be generated to construct 3D volumes other than 3D topographical models, can be generated for use in systems other than surgical navigation systems (e.g., for use in architectural modeling systems), and/or can be generated to conduct other activities besides surgery (e.g., medical or treatment planning). Furthermore, a person skilled in the art will understand (i) that the technology may have additional embodiments than illustrated in FIGS. 1-9 and (ii) that the technology may be practiced without several of the details of the embodiments described below with reference to FIGS. 1-9.

### A. Overview

As discussed above, virtual models of patients can be used for intraoperative guidance of surgery performed on the patients. In particular, the virtual models can be registered to the patients using surgical navigation systems and then displayed on a monitor for use by surgeons. To register the virtual models to the patients, the surgical navigation systems typically require volumetric datasets that are compliant with specific imaging standards. For example, many surgical navigation systems are configured to process, register, and display only volumetric datasets that are compliant with the DICOM Standard. Stated another way, these surgical navigation systems are unable to process, register, display, or otherwise use volumetric datasets that are not formatted in compliance with the DICOM Standard. Furthermore, many surgical navigation systems are typically employed in highly complex surgical cases requiring expensive and advanced imaging techniques, such as magnetic resonance imaging (MRI) or computed tomography (CT) imaging. Thus, volumetric datasets that are generated for use by surgical navigation systems are often produced from DICOM-compliant MRI or CT images that are captured with meta-data formatted in accordance with the DICOM Standard.

MRI or CT images, however, are not easily modified and do not easily allow for navigation against an intended change to the patient's anatomy. Furthermore, advanced imaging techniques are not required for many surgeries. For example, in some aesthetic surgery procedures (e.g., rhinoplasty), relatively inexpensive 3D topographical imaging is typically used in lieu of MRI, CT, or other more expensive and advanced imaging techniques. In these surgeries, progression towards a desired surgical outcome is often assessed through visual assessment of the 3D topographical images. Thus, using a surgical navigation system in these surgeries could prove useful in providing a surgeon more precise preoperative and intraoperative topographical navigation against an intended change to the patient' topographical anatomy (e.g., against the patient's actual topographical anatomy as captured in the 3D topographical images). Nevertheless, 3D object files (e.g., STL or OBJ 3D object files) generated from such 3D topographical images often do not comply with the specific imaging standards (e.g., the DICOM standard) that are required by the surgical navigation systems. As a result, the surgical navigation systems either (a) lack the ability to register the 3D topographical images to the patient or (b) merely incorporate data generated from the 3D topographical images into an already existing DICOM-compliant volumetric dataset that is based on other more expensive and advanced imaging (e.g., MRI, CT, etc.) of the patient.

To address these concerns, the inventors of the present technology have developed image processing methods for generating volumetric datasets (e.g., sequences of 2D cross-sectional images, 3D volumes reconstructed from the 2D cross-sectional images, etc.) from 3D topographical images of a patient and that comply with imaging standards that enable the volumetric datasets to be registered to the patient using surgical navigation systems. More specifically, several embodiments of the present technology involve obtaining one or more 3D topographical images of a patient (e.g., using a 3D imaging device), and generating one or more 3D models of the patient based, at least in part, on the 3D topographical image(s) of the patient. The 3D model(s) can include a composite 3D model including a first 3D topographical volume or mesh representing actual patient anatomy and/or a second 3D topographical volume or mesh representing desired patient anatomy (or a desired change to the patient's actual anatomy), as defined by a surgeon. The 3D model(s) and/or the 3D topographical volume(s) can be voxelated into one or more 3D voxelated volumes, the 3D voxelated volume(s) can be volume rendered into a single sequence of 2D cross-sectional images or multiple sequences of 2D cross-sectional images (e.g., with each sequence corresponding to a respective one of the 3D voxelated volumes). The 2D cross-sectional images can be processed to conform the sequences with a specific imaging standard (e.g., with the DICOM Standard).

In turn, the conformed sequence(s) of 2D cross-sectional images and/or 3D volumes reconstructed based, at least in part on, the conformed sequence(s) can be (i) used as a basis of registration to a patient and/or (ii) used for topographic navigation against a patient's true surface anatomy and/or against a desired change to the patient's true surface anatomy. In particular, volume datasets generated in accordance with various embodiments of the present technology can each depict one or more topographical layers of the original 3D topographical models, and can be overlayed or co-registered with a patient such that they can be used as a way for a surgeon to intraoperatively assess progress towards a desired surgical result by visually assessing a display (e.g., on a surgical navigation system) of a distance from a topographical contour representing the patient's true anatomy to a topographical contour curve representing the patient's desired anatomy.

Therefore, embodiments of the present technology can produce volumetric datasets from 3D topographical imaging of a patient and that can serve as a basis of registration to the patient for surgical navigation to allow a surgeon to assess progress towards a desired surgical outcome. As a result, the present technology can obviate the practice of obtaining MRI or CT imaging of the patient to generate volumetric datasets that can be registered to the patient using a surgical navigation system, which can reduce the cost of surgical operations (e.g., via use of relatively inexpensive 3D topographical imaging of the patient in lieu of MRI, CT, or other advanced imaging techniques) and can reduce patient exposure to radiation. Furthermore, the present technology can expand surgical navigation options to surgical procedures (e.g., aesthetic surgeries) that typically do not require MRI, CT, or other advanced imaging techniques.

### B. Selected Embodiments of Pressure Sensors, Including Pressure Sensors for APD Systems, and Associated Systems, Devices and Methods

FIG. 1 is a partially schematic representation of a modeling and navigation system 100 (“the system 100”) configured in accordance with various embodiments of the present technology. In some embodiments, the system 100 includes a DICOM-compliant patient modeling system configured to reconstruct 3D volumes or models and/or other images of a patient from 3D images taken of the patient. In other embodiments, the system 100 can include another modeling system configured to generate 3D models or volumes and/or other images of objects other than a patient.

As shown, the system 100 includes one or more imaging devices 101 (“3D cameras 101”), one or more user devices 105, one or more remote servers and/or databases 107, and a navigation system 110. In other embodiments, the user device(s) 105 and/or the remote server(s)/database(s) 107 can be omitted. Other well-known components of modeling systems are not illustrated in FIG. 1 or described in detail below so as to avoid unnecessarily obscuring aspects of the present technology.

The 3D camera(s) 101 can be any imaging device configured to generate three-dimensional images of an object. For example, the 3D camera(s) 101 can include a photogrammetric camera, such as a stereophotogrammetric camera. As another example, the 3D camera(s) 101 can include a 3D scanner, such as a 3D laser scanner or a 3D light (e.g., white light, structured light, infrared light, etc.) scanner. As a specific example, the 3D camera(s) 101 can include a Vectra® H1 Imaging System or a Vectra® H2 Imaging System commercially available from Canfield Scientific, Inc. of Parsippany, New Jersey. In operation, the 3D camera(s) 101 can be configured to generate one or more 3D images of an object, such as of a patient. For example, the 3D camera(s) 101 can be configured to generate one or more 3D images of a surface or topography of an object. As a specific example, the 3D camera(s) 101 can be configured to generate one or more 3D topographical images of patient anatomy, such as of soft tissue or skin of a patient's body (e.g., of a patient's face, breast, etc.). 3D images captured using the 3D camera(s) 101 can be stored as and/or used to generate 3D object files. The 3D object files can be stored in accordance with any suitable file format, such as STL, OBJ, IGES, STEP, MAX, FBX, 3DS, C4D, T2K, among other file formats.

The one or more user devices 105 can include personal computers, server computers, handheld or laptop devices, cellular or mobile telephones, wearable electronics, tablet devices, multiprocessor systems, microprocessor-based systems, set-top boxes, programmable consumer electronics, network PCs, minicomputers, mainframe computers, distributed computing environments that include any of the above systems or devices, or the like. In these and other embodiments, the one or more user devices 105 can include other remote or local devices, such as landline phones, fax machines, medical devices, thermostats, speakers, and other devices. As discussed in greater detail below, the one or more user devices 105 can include one or more processors and/or computer-readable media (e.g., software) configured to generate one or more 3D models based at least in part on 3D images obtained by the 3D camera(s) 101, voxelate the 3D model(s) into one or more 3D volumes, volume render the 3D voxelated volume(s) into one or more sequences of 2D cross-sectional images, process the 2D cross-sectional images of the sequence(s) to modify (e.g., change, alter, adjust, etc.) pixel values and/or perform other measurements on the image data, and/or conform the images to an appropriate imaging standard (e.g., to the DICOM Standard). In these and other embodiments, the remote server(s)/database(s) 107 and/or the navigation system 110 of the system 100 are configured to perform one or more of these functions in addition to or in lieu of the one of more user devices 105.

The user device(s) 105 can include memory and/or one or more databases. The memory and/or the one or more databases can store information, such as 3D images of an object (e.g., a patient) obtained by the 3D camera(s) 101, 3D models generated based at least in part on the 3D images, 3D voxelated volumes, sequences of pre-processed 2D cross-sectional images, sequences of post-processed 2D cross-sectional images, DICOM-compliant sequences of 2D cross-sectional images, health information, various alerts or warnings, user accounts/profiles, drivers/software necessary to operate certain applications and/or devices, and/or other information.

The remote server(s)/database(s) 107 can include an edge server which receives client requests and coordinates fulfillment of those requests through other servers. The remote server(s)/database(s) 107 can comprise computing systems. In these and other embodiments, the remote server(s)/database(s) can include a cloud server/database. Although the remote server(s)/database(s) 107 are displayed logically as a single server/database, the remote server(s)/database(s) 107 can be a distributed computing environment encompassing multiple computing devices and/or databases located at the same or at geographically disparate physical locations. In some embodiments, the remote server(s)/database(s) 107 correspond to a group of servers. As discussed above, the remote server(s)/database(s) 107 can include one or more processors and/or computer-readable media (e.g., software) configured to generate one or more 3D models based at least in part on 3D images obtained by the 3D camera(s) 101, voxelate the 3D model(s) into one or more 3D volumes, volume render the 3D voxelated volume(s) into one or more sequences of 2D cross-sectional images, process the 2D cross-section images of the sequence(s) to modify (e.g., change, alter, adjust, etc.) pixel values and/or perform other measurements on the image data, and/or conform the images to an appropriate imaging standard (e.g., to the DICOM Standard).

The remote server(s)/database(s) 107 can include memory and/or one or more databases. The memory and/or the one or more databases can warehouse (e.g. store) information, such as 3D images of an object (e.g., a patient) obtained by the 3D camera(s) 101, 3D models generated based at least in part on the 3D images, 3D voxelated volumes, sequences of pre-processed 2D cross-sectional images, sequences of post-processed 2D cross-sectional images, DICOM-compliant sequences of 2D cross-sectional images, health information, various alerts or warnings, user accounts/profiles, drivers/software necessary to operate certain applications and/or devices, and/or other information. In some embodiments, the one or more user devices 105, the remote server(s)/database(s) 107, and/or the navigation system 110 can each act as a server or client to other server/client devices.

The navigation system 110 can be any image-guided or model-guided system. For example, the navigation system 110 can be a surgical navigation system that enables computer-assisted surgery and/or tracking of medical instruments, such as a probe. Continuing with this example, the navigation system 110 can (a) track a location of a medical instrument (e.g., within an operating room) and/or a point of contact between the medical instrument and a patient, (b) display a representation of the instrument position within a volume, image, or model (e.g., of a patient), and/or (c) display information related to the location of the medical instrument or the point of contact.

As shown in FIG. 1, the navigation system 110 includes one or more processors 112, one or more displays 114, and one or more physical instruments 116 (e.g., probe(s)). In these and other embodiments, the navigation system 110 can include one or more computer-readable media (e.g., software), such as DICOM-compliant medical imaging software. The processor 112 can be configured to generate one or more 3D models based at least in part on 3D images obtained by the 3D camera(s) 101, voxelate the 3D model(s) into one or more 3D volumes, volume render the 3D voxelated volume(s) into one or more sequences of 2D cross-sectional images, process the 2D cross-section images of the sequence(s) to modify (e.g., change, alter, adjust, etc.) pixel values and/or perform other measurements on the image data, and/or conform the images to an appropriate imaging standard (e.g., to the DICOM Standard). In these and other embodiments, the processor 112 can be configured to register (a) imaging-standard-compliant sequences, one or more 2D cross-sectional images of the imaging-standard-compliant sequences, and/or 3D volumes reconstructed based at least in part on the imaging-standard-compliant sequences to (b) an object (e.g., to a patient in an operating room). In these and still other embodiments, the processor 112 can be configured to facilitate navigation (e.g., surgical navigation) using the imaging-standard-compliant sequences, one or more 2D cross-sectional images of the imaging-standard-compliant sequences, and/or 3D volumes reconstructed based at least in part on the imaging-standard-compliant sequences.

The physical instrument(s) 116 of the navigation system can include any suitable instrument. As described in greater detail below with respect to FIG. 9, the instrument(s) 116 can include a probe 116. In these and other embodiments, the instrument(s) 116 can include other devices, such as a scalpels, clamps, scissors, forceps, needles, retractors, suction instruments, scopes, staplers, catheters, and/or other devices (including non-medical instruments, such as a stylus).

The display 114 can be any suitable medium or screen configured to present information to an operator of the system 100 and/or an operator of the navigation system 110. For example, the display 114 can be a computer monitor, an LCD screen, an LED screen, a television, an augmented reality display or headset, a virtual reality display or headset, a mixed reality display or headset, an image projected against an object (e.g., a wall or screen, such as by a projector), and/or another suitable display. The navigation system 110 can include one or more user interfaces (e.g., one or more graphical user interfaces) that can be shown (e.g., displayed, depicted, projected, portrayed, etc.) on the display 114 (e.g., at the direction of the processor 112). For example, as described in greater detail below, the navigation system 110 can display 3D image(s), 3D model(s) generated based at least in part on the 3D image(s), 3D voxelated volumes, sequences of pre-processed 2D cross-sectional images, individual pre-processed 2D cross-sectional images, sequences of post-processed 2D cross-sectional images, individual post-processed 2D cross-sectional images, imaging-standard-compliant sequences of 2D cross-sectional images, individual imaging-standard-complaint 2D cross-sectional images, 3D volumes reconstructed based at least in part on one or more imaging-standard-compliant 2D cross-sectional images, and/or other information on the display 114. Continuing with this example, locations and/or points of contacts of the instrument(s) 116 can be tracked; representations of the instrument(s) 116 at the locations and/or points of contacts can be projected (e.g., overlayed, superimposed, blended, etc.) onto or within the registered imaging-standard-compliant 2D cross-sectional images and/or registered 3D volumes reconstructed based at least in part on the imaging-standard-compliant 2D cross-sectional images; and/or the registered imaging-standard-compliant 2D cross-sectional images, the registered reconstructed 3D volumes, the representations of the instrument(s) 116, and/or related information (e.g., a depth or distance between sub-volumes of the imaging-standard-compliant 2D cross-sectional images and/or of the reconstructed 3D volumes at the locations or points of contact of the instrument(s) 116) can be presented to an operator of the navigation system 110 on the display 114.

The components of the system 100 can communicate with one another over one or more networks 103, including public or private networks (e.g., the internet). The one or more networks 103 allow for communication within the system 100 and/or for communication with one or more devices outside of the system 100. The one or more networks 103 can include one or more wireless networks and/or messaging protocols, such as, but not limited to, one or more of a Near Field Communication (NFC) Network, a Local Area Network (LAN), Wireless Local Area Network (WLAN), a Personal Area Network (PAN), Campus Area Network (CAN), a Metropolitan Area Network (MAN), a Wide Area Network (WAN), a Wireless Wide Area Network (WWAN), Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 3.75G, 4G, 5G, LTE networks, enhanced data rates for GSM evolution (EDGE), General Packet Radio Service (GPRS), enhanced GPRS, TCP/IP, SMS, MMS, extensible messaging and presence protocol (XMPP), real time messaging protocol (RTMP), instant messaging and presence protocol (IMPP), instant messaging, USSD, IRC, or any other wireless data networks or messaging protocols. Network(s) 103 may also include wired networks.

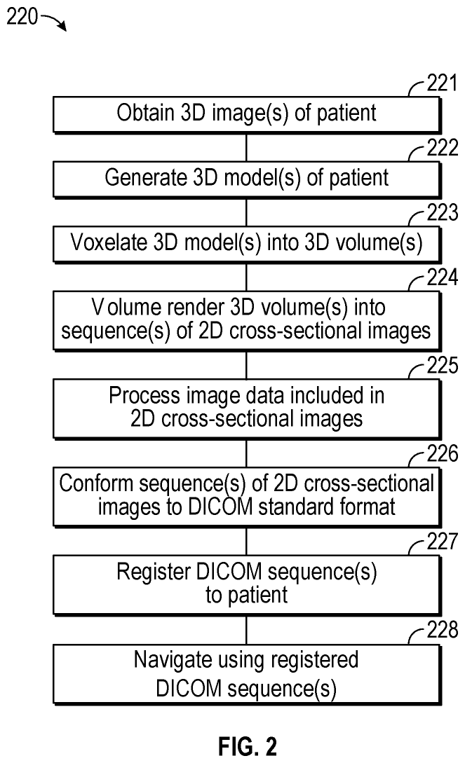

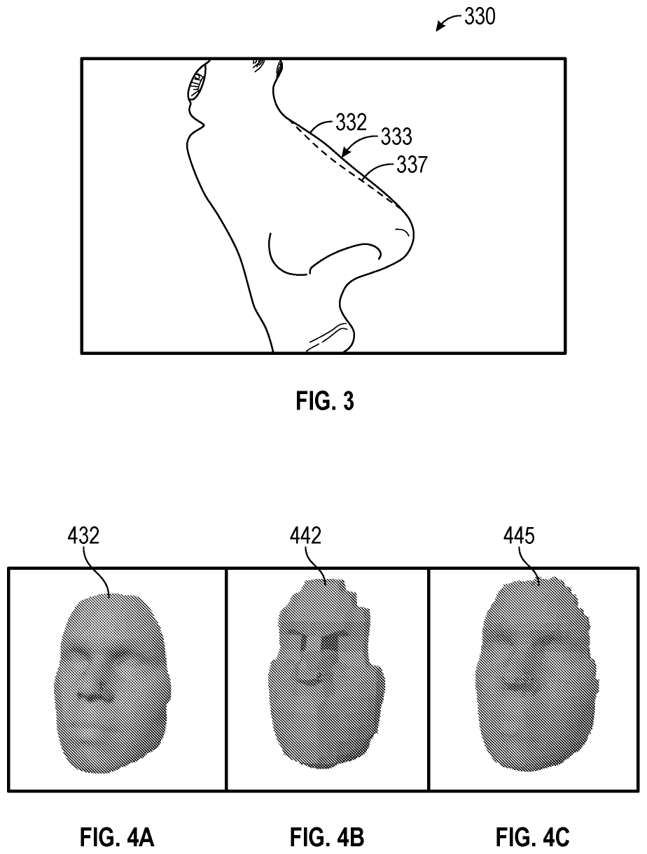

FIG. 2 is a flow diagram illustrating a method 220 of generating and using image data, such as DICOM-compliant image data for surgical navigation, in accordance with various embodiments of the present technology. The method 220 is illustrated as a set of blocks, steps, operations, or processes 221-228. All or a subset of the blocks 221-228 can be executed at least in part by various components of a modeling and/or navigation system, such as the modeling and navigation system 100 of FIG. 1. For example, all or a subset of the blocks 221-228 can be executed at least in part by one or more 3D cameras (e.g., the 3D camera 101 of FIG. 1), one or more user devices (e.g., the user device 105 of FIG. 1), one or more remote servers or databases (e.g., the remote server(s)/database(s) 107 of FIG. 1), and/or one or more components of a navigation system (e.g., the processor(s) 112, the display(s) 114, and/or the physical instrument(s) 116 of the navigation system 110 of FIG. 1). Additionally, or alternatively, all or a subset of the blocks 221-228 can be executed at least in part by an operator (e.g., a user, a patient, a surgeon, a physician, a nurse, etc.) of the system. Furthermore, any one or more of the blocks 221-228 can be executed in accordance with the discussion above. Many of the blocks 221-228 of the method 220 are discussed in detail below with reference to FIGS. 3-9 for the sake of clarity and understanding.

The method 220 begins at block 221 by obtaining one or more 3D images of a patient. In some embodiments, the 3D images of the patient can be obtained using one or more imaging devices and/or optical sensors, such as one or more 3D cameras and/or one or more 3D scanners. In these and other embodiments, the 3D images of the patient can be 3D images of soft tissue or skin of the patient. For example, the 3D images of the patient can be 3D topographical images of a patient's face, breast, or other part of the patient's skin or body. The 3D images captured or obtained at block 221 can be stored as and/or used to generate one or more 3D object files. The 3D object files can be stored in accordance with any suitable file format, such as STL, OBJ, IGES, STEP, MAX, FBX, 3DS, C4D, T2K, among other file formats.

At block 222, the method 220 continues by generating one or more 3D models of the patient. The 3D model(s) can be based at least in part on the 3D images of the patient captured or obtained at block 221. Generating the 3D models can include (a) generating a 3D model representing a patient's true or actual anatomy and/or (b) generating a 3D model representing a patient's planned or desired anatomy. The patient's desired anatomy can be defined at least in part by an operator of the system, such as a surgeon or a physician. As a specific example, generating the 3D models can include generating a composite 3D model of the patient that includes (i) a first 3D topographical volume (also referred to as a 3D topographical mesh) representing one or more contours of a patient's existing or actual anatomy and (ii) a second 3D topographical volume representing one or more contours of a patient's desired anatomy (e.g., defined by a surgeon or another operator of the system). In some embodiments, the one or more 3D models of the patient can be generated, at least in part, using image editing software. For example, the one or more 3D models can be generated using Mirror® Medical Imaging Software commercially available from Canfield Scientific, Inc. of Parsippany, New Jersey. Further details regarding obtaining 3D images of a patient and/or generating 3D models of the patient that include representations of a patient's actual and/or desired anatomy are provided in U.S. Pat. No. 10,810,799, which is incorporated by reference herein in its entirety.

For the sake of clarity and understanding, consider FIG. 3 that illustrates a display of a composite 3D topographical model 330 of a patient (e.g., of the patient's nose) configured in accordance with various embodiments of the present technology. As shown, the topographical model 330 includes a first 3D topographical mesh 332 representing a contour of the patient's actual patient anatomy and a second 3D topographical mesh 337 representing a contour of the patient's desired patient anatomy (or a desired change to the patient's actual anatomy, such as a desired change defined by a surgeon based at least in part on the patient's actual anatomy). The first 3D topographical mesh 332 can be referred to as a first sub-volume of the composite 3D topographical model 330, and the second 3D topographical mesh 337 can be referred to as a second sub-volume of the composite 3D topographical model 330. In the illustrated embodiment, the patient's actual anatomy includes a dorsal hump 333 near the center of the patient's nose, which is represented by the first 3D topographical mesh 332 in the composite 3D topographical model 330. By contrast, the patient's desired anatomy omits the dorsal hump 333, representing a desired removal of the dorsal hump 333 from the patient's actual anatomy. Thus, the second 3D topographical mesh 337 is different from or diverges from the first 3D topographical mesh 332 generally at the location of the dorsal hump 333 within the composite 3D topographical model 330, and differences between the first 3D topographical mesh 332 and the second 3D topographical mesh 337 at this location represent magnitudes of desired changes to the patient's existing or actual anatomy at the corresponding location on the patient. In the illustrated embodiment, the second 3D topographical mesh 334 can largely agree or align with the first 3D topographical mesh 332 at locations other than at the location of the dorsal hump 333.

As discussed above and in greater detail below, the 3D images obtained at block 221 and/or the 3D model(s) of the patient generated at block 222 of the method 220 may not be (e.g., at least when initially obtained or generated) compliant with certain desired imaging standards, such as the DICOM Standard. As a result, navigation systems (e.g., surgical navigation systems) that are configured to process and/or display image data of a particular imaging standard (e.g., the DICOM Standard) may be unable to process and/or display the 3D images obtained at block 221 and/or the 3D model(s) of the patient generated at block 222. Thus, blocks 223-226 of the method 220 are generally directed to conforming image data from blocks 221 and/or 222 to various imaging standards. For the sake of clarity and understanding, blocks 223-228 of the method 220 are discussed in detail below in the context of conforming image data to the DICOM Standard and performing surgical navigation using a surgical navigation system. A person of ordinary skill in the art, however, will recognize and appreciate that the present technology can be applied in other contexts, such as to conform imaging data to another imaging standard, to perform another type of navigation, and/or to use a different type of navigation system. Such other contexts are within the scope of the present technology and this disclosure.

At block 223 of FIG. 2, the method 220 continues by voxelating the 3D model(s) generated at block 222 into one or more 3D voxelated volumes (also referred to as 3D voxelated meshes). In some embodiments, the 3D models or 3D sub-volumes/topographical volumes of a 3D model are voxelated separately or independently from one another. For example, referring again to FIG. 3, the first 3D topographical mesh 332 representing actual patient anatomy can be voxelated into a first 3D voxelated mesh, and the second 3D topographical mesh 337 representing desired patient anatomy can be separately or independently voxelated into a second 3D voxelated mesh.

In some embodiments, a 3D voxelated mesh generated at block 223 can be smoothed by subjecting the 3D voxelated mesh to a smoothing algorithm to, for example, increase resolution of the 3D voxelated mesh. For example, using a voxelating algorithm and/or a smoothing algorithm built into open source software known as Blender, a 3D topographical mesh can be voxelated into a 3D voxelated mesh, and the 3D voxelated mesh can be smoothed into a smoothed 3D voxelated mesh. In other embodiments, other voxelating software and/or other smoothing software can be used.

For the sake of clarity and understanding, consider FIGS. 4A-4C. FIG. 4A illustrates a display of a 3D topographical mesh 432 generated at block 222 of FIG. 2 in accordance with various embodiments of the present technology. FIG. 4B illustrates a display of a 3D voxelated mesh 442 generated by voxelating the 3D topographical mesh 432 of FIG. 4A at block 223 of FIG. 2 in accordance with various embodiments of the present technology. FIG. 4C illustrates a display of a smoothed 3D voxelated mesh 445 generated by subjecting the 3D voxelated mesh 442 of FIG. 4B to a smoothing algorithm at block 223 of FIG. 2 in accordance with various embodiments of the present technology.

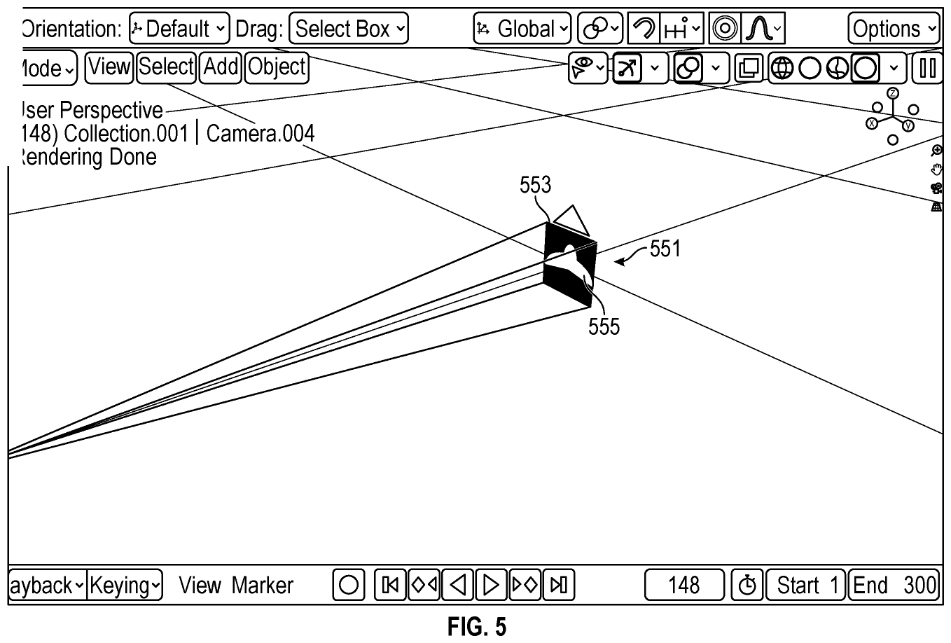

At block 224 of FIG. 2, the method 220 continues by volume rendering the 3D voxelated volume(s) into one or more sequences of 2D cross-sectional images. In some embodiments, volume rendering a 3D voxelated volume can include advancing a frame through the 3D voxelated volume at equally spaced, non-overlapping intervals to generate a series of sequential, non-overlapping slices of the 3D voxelated volume. FIG. 5 is a display of a 2D cross-sectional image 551 generated by advancing a frame 553 through a 3D voxelated volume (e.g., the 3D voxelated mesh 442 of FIG. 4B or the smoothed voxelated mesh 445 of FIG. 4C) as part of the volume rendering process performed at block 224 of FIG. 2 in accordance with various embodiments of the present technology. As shown in FIG. 5, the 2D cross-sectional image 551 includes a 2D slice 555 of the 3D voxelated mesh at the location of the frame 553.

In some embodiments, the volume rendering can be performed using ray casting or any other suitable rendering algorithm that produces appropriate 2D images of the 3D voxelated volume. For example, the volume rendering can be performed using open source software known as Blender. More specifically, the volume rendering can be performed using a ray-cast algorithm and/or a shader script of Blender. The shader script can be a script written in a node-based shader language of Blender. In other embodiments, other volume rendering software and/or other script software can be used to volume render one or more 3D voxelated volumes at block 224.

As discussed above, the volume rendering process performed at block 224 produces a sequence or series of pre-processed 2D cross-sectional images that include 2D slices of a 3D voxelated volume that each describe (or together define) the 3D voxelated volume. Each of the pre-processed 2D cross-sectional images can include a number of pixels arranged in rows and columns. Information contained in each pixel of a pre-processed 2D cross-sectional image can include a 16-bit (or other length) value representing an integer value, for example, between 0 and 65,535. The integer value can correspond to a color on the grayscale or other color space. Information contained in each pixel of a pre-processed 2D cross-sectional image can be used to identify the corresponding 3D voxelated volume and/or to later assign each pixel another (e.g., more accurate) color value (as described in greater detail below at block 225). In other words, the value assigned to each pixel of a pre-processed 2D cross-sectional image during the volume rendering process can depend, at least in part, on the 3D voxelated volume that the pre-processed 2D cross-sectional image describes. For example, pixels of a first pre-processed 2D cross-sectional image (e.g., pixels that are included in a corresponding 2D slice) of a first 3D voxelated volume can be assigned pixels values in a first range of values. Continuing with this example, pixels of a second pre-processed 2D cross-sectional image (e.g., pixels that are included in a corresponding 2D slice) of a second 3D voxelated volume (different from the first 3D voxelated volume) can be assigned pixel values in a second range of values. Pixel values of the second range can be different from pixel values of the first range. Thus, the values assigned to the pixels in each of the first and second pre-processed 2D cross-sectional images can be used to identify which of the 3D voxelated volumes (e.g., the first 3D voxelated volume or the second 3D voxelated volume) the pixels of the first and second pre-processed 2D cross-sectional images describe. Thus, the values assigned to the pixels of a pre-processed 2D cross-sectional image can be used to uniquely identify the corresponding 3D voxelated volume.

In some embodiments, individual 3D voxelated volumes can be volume rendered separately or independently at block 224. For example, a first 3D voxelated volume generated at block 223 (e.g., a 3D voxelated volume generated from the 3D topographical mesh 332 of FIG. 3) and representing actual patient anatomy can be volume rendered into a first sequence of pre-processed 2D cross-sectional images at block 224. As part of the volume rendering process, pixels in each pre-processed 2D cross-sectional image of the first sequence can be assigned a first range of integer pixel values corresponding to a first range of colors on the greyscale or other color scale. Continuing with this example, a second 3D voxelated volume generated at block 223 (e.g., a 3D voxelated volume generated from the 3D topographical mesh 337 of FIG. 3) and representing desired patient anatomy can be separately or independently volume rendered into a second sequence of pre-processed 2D cross-sectional images at block 224. As part of the volume rendering process, pixels in each pre-processed 2D cross-sectional image of the second sequence can be assigned a second range of integer pixel values corresponding to a second range of colors on the greyscale or other color scale. Thus, in the above example, the volume rendering process performed at block 224 can produce two or more separate sequences of pre-processed 2D cross-sectional images, with each sequence corresponding to a respective one of the two or more 3D voxelated volumes generated at block 223.

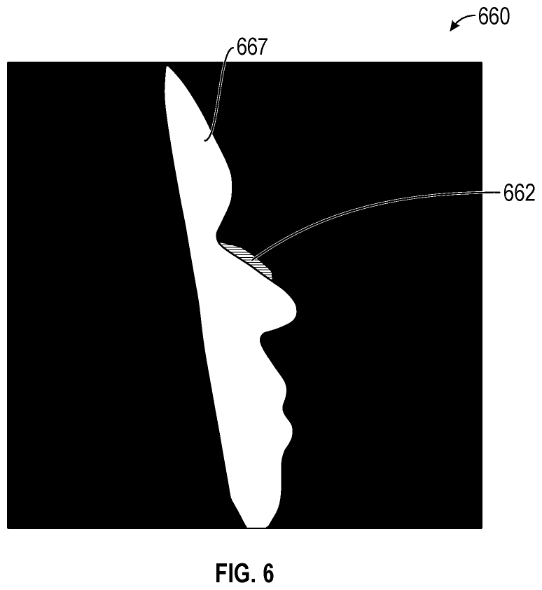

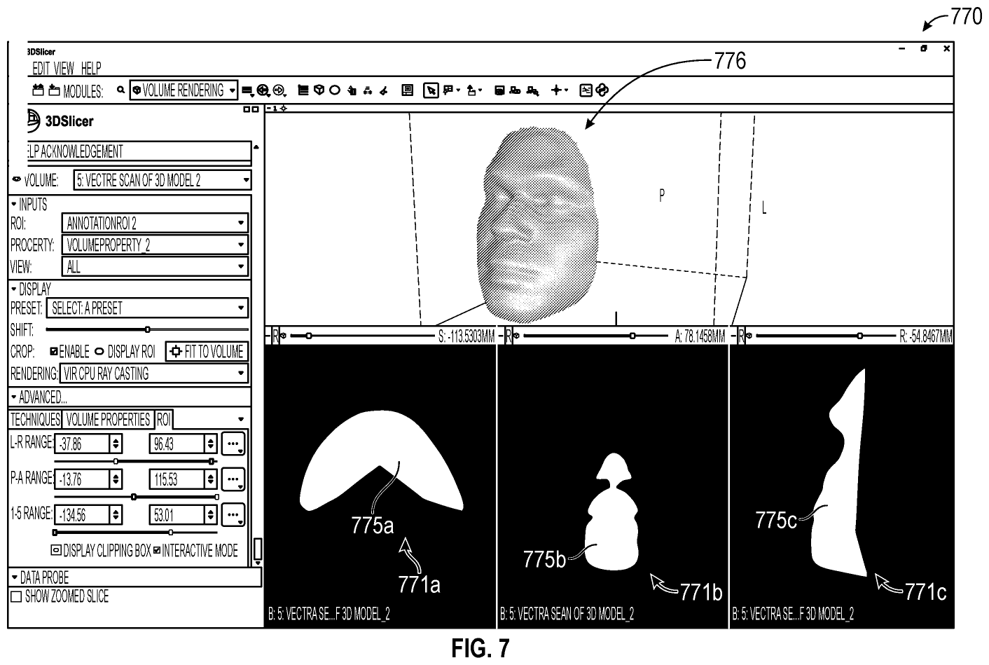

Additionally, or alternatively, two or more separate or different 3D voxelated volumes from block 223 can be volume rendered together at block 224 into a single sequence of pre-processed 2D cross-sectional images. In some embodiments, volume rendering the two or more separate or different voxelated volumes can include (a) blending the images at the time of rendering using alpha blending or (b) using another suitable technique, to generate a single sequence of pre-processed 2D cross-sectional images. For example, FIG. 6 is a display of a pre-processed 2D cross-sectional image 660 generated by volume rendering two 3D voxelated volumes into a single sequence of pre-processed 2D cross-sectional images in accordance with various embodiments of the present technology. The two 3D voxelated volumes used to generated the pre-processed 2D cross-sectional image 660 of FIG. 6 can include a first 3D voxelated volume of actual patient anatomy (e.g., a 3D voxelated volume corresponding to the 3D topographical mesh 332 of FIG. 3) and a second 3D voxelated volume of desired patient anatomy (e.g., a 3D voxelated volume corresponding to the 3D topographical mesh 337 of FIG. 3). Thus, as shown in FIG. 6, the pre-processed 2D cross-sectional image 660 can include (i) a first 2D slice 662 of the first 3D voxelated volume representing actual patient anatomy, and (ii) a second 2D slice 667 of the second 3D voxelated volume representing desired patient anatomy (or a desired change to the actual patient anatomy). Because the first image slice 662 describes the first 3D voxelated volume, the volume rendering process has assigned pixels included in the 2D slice 662 integer values that fall within a first range of integer values corresponding to a range of grey colors (shown via crosshatching in FIG. 6) on the greyscale or other color space. In addition, because the second image slice 667 describes the second 3D voxelated volume, the volume rendering process has assigned pixels included in the 2D slice 667 integer values falling within a second range of integer values corresponding to a range of white colors on the greyscale or other color space. In the event that a pixel of the pre-processed 2D cross-sectional image 660 describes both the first 3D voxelated volume and the second 3D voxelated volume, the volume rendering process can assign the pixel a value that falls within a third range of integer values (e.g., different from the first and/or the second range of integer values) corresponding to a range of colors on the greyscale or other color space. The value assigned to the pixel can be a summation, an average, a different, or another logical relation of the pixel values assigned to the pixels included in the 2D slice 662 and the pixel values assigned to the pixels included in the 2D slice 667.

In some embodiments, voxelating the 3D model(s) (block 223) and/or volume rendering the 3D voxelated volume(s) (block 234) can include performing measurements on the 3D voxelated volumes generated at block 223. For example, processing the image data can include performing measurements to determine a length in real world units of a 3D voxelated volume generated at block 223. As another example, processing the image data can include performing measurements to determine a height and/or a width in real world units of a 3D voxelated volume. The measurements can be performed digitally in the 3D modeling space and/or in the 3D rendering environment. For example, the measurements can be performed using a digital ruler tool of open source software known as Blender or another digital measuring software. As discussed in greater detail below with respect to block 226, the measurements can be used to reverse calculate dimension attributes for DICOM files (e.g., for DICOM meta-data files).

At block 225, the method 220 continues by processing image data included in the 2D cross-sectional images generated at block 224. As discussed above, a pixel value assigned to a pixel of a 2D cross-sectional image generated at block 224 depends, at least in part, on which 3D voxelated volume from block 223 the pixel describes. Thus, pixel values assigned during the volume rendering process of block 224 are not necessarily in pixel value ranges that are optimal for viewing on a display of DICOM-compliant medical imaging software. Therefore, processing image data at block 225 can include digitally processing (e.g., using Python) the 2D cross-sectional images generated at block 224, pixel-by-pixel, to update pixel values to new values that are consistent with values (a) that are commonly used in MRI or CT images, (b) that conform to the DICOM Standard, and/or (c) that optimize the 2D cross-sectional images for viewing on a display of DICOM-compliant medical imaging software (e.g., to better visualize differences between composite layers of a reconstructed 3D model).

Pixel values of a pre-processed 2D cross-sectional image can be read as raw image data and converted to a list of individual pixel values. For example, individual pixels of a pre-processed 2D cross-sectional image can be read and then assigned a new value in a corresponding post-processed 2D cross-sectional image that depends, at least in part, on its value in the pre-processed 2D cross-sectional image. Continuing with this example, if a pixel of a pre-processed 2D cross-section image was assigned a pixel value during volume rendering that falls within a high range of values, that pixel can be assigned value “A” in the corresponding post-processed 2D cross-sectional image. On the other hand, if the pixel of the pre-processed 2D cross-sectional image was assigned a pixel value during volume rendering that falls within a low range or a middle range, that pixel can be assigned value “C” or value “B,” respectively, in the corresponding post-processed 2D cross-sectional image.

In some embodiments, the values “A,” “B,” and “C” of the above example can be 16-bit unsigned integer values in ranges consistent with different tissue types as described in the DICOM Standard for grayscale images. Additionally, or alternatively, the values “A,” “B,” and “C” can correspond to new colors that are used to identify which of the 3D voxelated volumes from block 223 a pixel describes. For example, pixels of a post-processed 2D cross-sectional image that are included in a 2D slice of a first 3D voxelated volume from block 223 can be assigned value “A” at block 225. Continuing with this example, pixels of a different post-processed 2D cross-sectional image that are included in a 2D slice of a second 3D voxelated volume from block 223 can be assigned value “B” or value “C” at block 225. As another example, pixels of a post-processed 2D cross-sectional image that are included in a 2D slice of a first 3D voxelated volume from 223 can be assigned value “A” at block 225, pixels of the same post-processed 2D cross-sectional image but included in a 2D slice of a second 3D voxelated volume from block 223 can be assigned value “B” at block 225, and pixels of the same post-processed 2D cross-sectional image that are included in both the 2D slice of the first 3D voxelated volume from block 223 and the 2D slice of the second 3D voxelated volume from block 223 can be assigned value “C” at block 225. The new colors corresponding to the values “A,” “B,” and “C” can be selected to improve or better highlight a contrast or difference (a) between 2D slices of a first 3D voxelated volume and 2D slices of a second 3D voxelated volume, and/or (b) between 3D volumes reconstructed from the 2D slices of the first 3D voxelated volume and 3D volumes reconstructed from the 2D slices of the second 3D voxelated volume.

In some embodiments, the volume rendering process of block 224 (e.g., as opposed to the processing performed at block 225) can assign pixel values to pixels of a pre-processed 2D cross-sectional image that correspond to colors (a) that are consistent with colors typically found in MRI or CT images, (b) that are consistent with ranges of different tissue types as described in the DICOM Standard for greyscale images, and/or (c) that are optimized for viewing the 2D cross-sectional images on a display of DICOM-compliant medical imaging software. In these embodiments, updating the pixel values assigned to pixels of the 2D cross-sectional images during processing of the image data at block 225 can be omitted from the method 220.

As discussed above with respect to block 224, the volume rendering process can be performed separately or independently on two or more separate or different 3D voxelated volumes to produce two or more sequences of pre-processed 2D cross-sectional images. In these embodiments, the pre-processed 2D cross-sectional images of each sequence can be processed at block 225 separately or independently from the pre-processed 2D cross-sectional images of other sequences. For example, the image data of pre-processed 2D cross-sectional images of a first sequence can be processed at block 225 to update the pixels values in the pre-processed 2D cross-sectional images of the first sequence. Continuing with this example, the image data of pre-processed 2D cross-sectional images of a second sequence can be independently or separately processed at block 225 to update the pixel values in the pre-processed 2D cross-sectional images of the second sequence.

As also discussed above with respect to block 224, the volume rendering process can include volume rendering two or more separate or different 3D voxelated volumes together into a single sequence of 2D cross-sectional images. In these embodiments, processing the image data of the pre-processed 2D cross-sectional images of the single sequence can include (e.g., simultaneously) (a) updating the pixel values of pixels in the pre-processed 2D cross-sectional images describing a first 3D voxelated volume, and (b) updating the pixel values of pixels in the pre-processed 2D cross-sectional images describing a second 3D voxelated volume.

At block 226, the method 220 continues by processing the sequence(s) of 2D cross-sectional images from blocks 224 and/or 225 to conform the sequence(s) to the DICOM Standard. Typically, DICOM-compliant MRI or CT images include meta-data that is captured and formatted at the time the MRI or CT images are taken. The meta-data can reflect various information relating to an MRI or CT image, such as the method used to generate the MRI or CT image, the machine used to capture the MRI or CT image, and/or an identifier of the patient who is the subject of the MRI or CT image. Other information in the meta-data of an MRI or CT image can include pixel spacing, slice thickness, image position relative to the patient, slice location, and/or various unique identifiers required by the DICOM Standard. Much of the information included in the meta-data of an MRI or CT image is (a) descriptive in that the information is intrinsic to a particular machine and/or method used to capture the MRI or CT image and (b) required by the DICOM Standard.

By contrast, the 3D images captured at block 221, the 3D models at block 222, the 3D voxelated volumes at block 223, and/or the 2D cross-sectional images at blocks 224 and/or 225 can (a) omit or lack some of the meta-data information required by the DICOM Standard and/or (b) include meta-data information in a formatting that is not compliant with the DICOM Standard. Thus, at block 226, the method 220 can (e.g., using Python) generate meta-data information required by the DICOM Standard, format or reformat meta-data information in a manner that is compliant with the DICOM Standard, and/or otherwise process the sequence(s) of 2D cross-sectional images to convert them into DICOM-compliant sequence(s) (“DICOM sequence(s)”). For example, the method 220 can use the measurements performed at block 223 and/or block 224 to calculate various DICOM dimensional attributes, such as pixel spacing, slice thickness, image position relative to the patient, and/or slice location. Additionally, or alternatively, the method 220 can capture and/or generate various unique identifiers (e.g., a method of capturing identifier, a machine identifier, a patient identifier, etc.) that are required by the DICOM Standard. The meta-data attributes and/or the unique identifiers can be populated into data fields of a DICOM-compliant file (e.g., an MRI or CT meta-data file template) to, for example, ensure that the meta-data information is formatted in compliance with the DICOM Standard. In these and other embodiments, the method 220 can process the 2D cross-sectional images of the sequence(s) such that the 2D cross-sectional images have pixel padding that is consistent with the DICOM Standard.

The results of the processing performed at block 226 include DICOM-compliant sequence(s) of 2D cross-sectional images having meta-data files populated with values that are calculated by the method 220 to enable DICOM-compliant medical imaging software to, using the 2D cross-sectional images of the DICOM sequence(s), reconstruct and/or display a 3D volume that appropriately and/or accurately describes dimensions of a patient's (e.g., actual and/or desired) anatomy. For example, the results of the processing performed at block 226 can include one or more DICOM-compliant volumetric datasets (a) that describe a first volume in which surface contours of the first volume match the topographical contours of the patient's actual anatomy, and/or (b) that describe a second volume in which surface contours of the second volume match the topographical contours of a desired change to a patient's actual anatomy. In other words, the method 220 (i) starts with a volume (e.g., a 3D model or a 3D topographical volume at block 222, and/or a 3D voxelated volume at block 223) and (ii) reverse-constructs DICOM dimensional values and other information from 2D cross-sectional images of that volume at blocks 224-226 such that DICOM-compliant medical imaging software can reconstruct a volume that is an accurate depiction of the patient.

In some embodiments, the results of the processing performed at block 226 can be uploaded and/or used by a surgical navigation system or another navigation system to display various information included in the DICOM sequence(s). For the sake of clarity and understanding, consider FIG. 7 that illustrates various information that can be displayed (e.g., in a user interface 770 and/or on a display) using (a) DICOM-compliant medical imaging software running on surgical navigation system and/or (b) a DICOM sequence generated at block 226, in accordance with various embodiments of the present technology. In particular, the DICOM-compliant medical imaging software can process and/or display various 2D cross-sectional images 771a-771c of a DICOM sequence that include 2D slices 775a-775c, respectively, of a 3D voxelated volume from block 223. In these and other embodiments, the DICOM-compliant medical imaging software can process 2D cross-sectional images 771 of the DICOM sequence and reconstruct a 3D volume 776. The reconstructed 3D volume 776 can appropriately and/or accurately describe dimensions of a patient's anatomy, such as the patient's actual anatomy and/or a desired change to the patient's actual anatomy. Additionally, or alternatively, topographical/surface contours of the reconstructed 3D volume 776 can match topographical/surface contours of (i) the patient's actual or true anatomy and/or (ii) one or more desired changes to the patient's actual anatomy. In some embodiments, the DICOM-compliant medical imaging software can display the reconstructed 3D volume 776 (e.g., in the user interface 770 and/or on a display) in addition to or in lieu of a display of one or more of the corresponding 2D cross-sectional images 771.

As discussed above, the volume rendering process of block 224 can be performed separately or independently on two or more separate or different 3D voxelated volumes to produce two or more sequences of pre-processed 2D cross-sectional images, and image data of each sequence can be separately or independently processed at block 225. In these embodiments, the two or more sequences of 2D cross-sectional images can be separately or independently processed at block 226 to conform the sequences to the DICOM Standard. Thus, the results of the processing at block 226 can include two or more DICOM sequences that each correspond to (a) a respective one of the two or more sequences from blocks 224 and/or 225, and/or (b) to a respective one of the 3D voxelated volumes from block 223.

As also discussed above, the volume rendering process of block 224 can include volume rendering two or more separate or different 3D voxelated volumes together into a single sequence of 2D cross-sectional images, and image data of the single sequence can be processed at block 225. In these embodiments, the single sequence of 2D cross-sectional images can be further processed at block 226 to conform the single sequence to the DICOM Standard. Thus, the results of the processing at block 226 can include a single DICOM sequence that corresponds to (a) the single sequence from blocks 224 and/or 225, and/or (b) multiple 3D voxelated volumes from block 223. 3D volumes reconstructed from the single DICOM sequence by DICOM-compliant medical imaging software can, for example, represent topographical/surface contours of both (i) a patient's actual or true anatomy and (ii) one or more desired changes to the patient's actual anatomy. Sub-volumes (e.g., defined at least in part by differing pixel values) included in the reconstructed 3D volumes can represent a difference between the patient's actual anatomic contours and desired changes to the anatomic contours at the corresponding location on the patient.

At block 227, the method 220 continues by registering the DICOM sequence(s) to the patient. In some embodiments, the DICOM sequence(s) are registered to the patient in an operating room and/or using a surgical navigation system or another navigation system. In these and other embodiments, the DICOM sequence(s) can be registered to the patient using any appropriate registration modality, such as photometric, infrared, electromagnetic, or another suitable modality. In these and still other embodiments, the DICOM sequence(s) can be registered to the patient such that topographical/surface contours (e.g., topographical/surface contours representing a patient's actual anatomy) in 3D volumes reconstructed from the DICOM sequence(s) are level with corresponding topographical/surface contours on the patient (e.g., on the patient's skin). Additionally, or alternatively, the DICOM sequence(s) can be registered to the patient such that a bulk of a 3D volume reconstructed from the DICOM sequence(s) is within or internal to the patient (e.g., within, beneath, or internal to the patient's skin). In these and other embodiments, registering the DICOM sequence(s) to the patient can include registering (e.g., individual ones or a subset of) 2D cross-sectional images of a DICOM sequence to the patient.

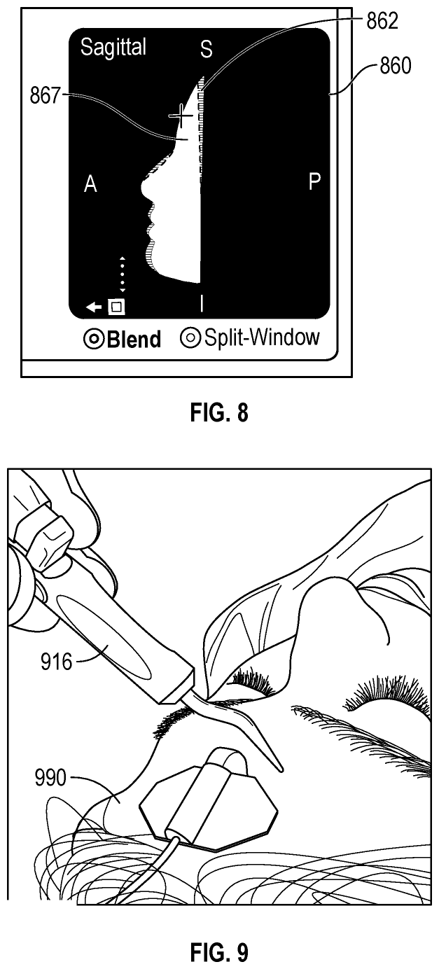

In embodiments in which there are multiple DICOM sequences (e.g., a first DICOM sequence corresponding to a first 3D voxelated volume representing actual patient anatomy and a second DICOM sequence corresponding to a second 3D voxelated volume representing a desired change to the actual patient anatomy), the method 220 can register a first 3D volume reconstructed from one of the DICOM sequences to the patient and then register or overlay (e.g., onto or against the first 3D volume) a second 3D volume reconstructed from another of the DICOM sequences. For the sake of clarity and understanding, consider FIG. 8 that illustrates a display 860 of two 2D slices 862 and 867 registered to a patient in accordance with various embodiments of the present technology. The 2D slice 862 can be included in a 2D cross-sectional image of a first DICOM sequence that corresponds to a first 3D voxelated volume representing actual patient anatomy, and the 2D slice 867 can be included in a 2D cross-sectional image of a second DICOM sequence that corresponds to a second 3D voxelated volume representing desired patient anatomy. In some embodiments, the method 220 can register the 2D slices 862 and 867 to the patient by (a) registering one of the 2D slices 862, 867 (e.g., the 2D slice 862) to the patient, and (b) overlaying the other of the 2D slices 862, 867 (e.g., the 2D slice 867) onto or against the one of the 2D slices 862, 867 (e.g., the 2D slice 862), for example, using a best fit algorithm or another suitable algorithm. Alternatively, the method can (a) register a first 3D volume (not shown) reconstructed based at least in part on one of the 2D slices 862, 867 (e.g., the 2D slice 862) to the patient, and (b) overlay a second 3D volume (not shown) reconstructed based at least in part on the other of the 2D slices 862, 867 (e.g., the 2D slice 867) onto or against the first reconstructed 3D volume, for example, using a best fit algorithm or another suitable algorithm. In still other embodiments, the method 220 can register the 2D slices 862 and 867 to the patient by (1) registering the 2D slice 862 and/or a first 3D volume reconstructed based at least in part on the 2D slice 862 to the patient and (2) registering the 2D slice 867 and/or a second 3D volume reconstructed based at least in part on the 2D slice 867 to the patient independent of the registration of the 2D slice 862 and/or the first reconstructed 3D volume to the patient. In any of the above embodiments, two 2D slices 862 and 867 (or two corresponding, reconstructed 3D volumes) can be simultaneously displayed after registration, as shown in FIG. 8. Any difference shown on the display 860 between the 2D slices 862 and 867 at a region of interest (e.g., at the center of the patient's nose) can represent the difference between the patient's actual or real anatomy and a desired anatomy (e.g., a desired surgical result).

In embodiments in which there is a single DICOM sequence (e.g., a DICOM sequence of 2D cross-sectional images having both 2D slices of a first 3D voxelated volume representing actual patient anatomy and 2D slices of a second 3D voxelated volume representing a desired change to the actual patient anatomy), the method 220 can register the single DICOM sequence to the patient by registering a portion (e.g., a 2D slice included in a 2D cross-sectional image of the DICOM sequence and/or a portion of a 3D volume reconstructed from the DICOM sequence) of the DICOM sequence that corresponds to actual patient anatomy to the patient. For example, in a case in which desired patient anatomy represents a removal of actual patient anatomy, the method 220 can register outermost topographic/surface contours (e.g., at a region of interest) of the reconstructed 3D model (e.g., that is/are identified using pixel values, coordinates of the pixels in the 2D cross-sectional images, a boundary fill algorithm, or another suitable method) to the patient's real anatomy. Thus, in this example, topographical/surface contours (i) of the 3D model reconstructed from the single DICOM sequence and (ii) that correspond to a patient's actual anatomy can be level with corresponding topographical/surface contours (e.g., at a region of interest) on the patient after registration. Additionally, or alternatively, a bulk of the reconstructed 3D volume (representing a difference between the topographical/surface contours corresponding to actual patient anatomy and topographical/surface contours of desired patient anatomy) can be internal to (e.g., within, beneath, behind) the patient's skin after registration.

As another example, in a case in which desired patient anatomy represents an addition to actual patient anatomy, the method 220 can register innermost topographic/surface contours (e.g., at a region of interest) of the reconstructed 3D model (e.g., that is/are identified using pixel values, coordinates of the pixels in the 2D cross-sectional images, a boundary fill algorithm, or another suitable method) to the patient's real anatomy. Thus, in this example, topographical/surface contours (i) of the 3D model reconstructed from the single DICOM sequence and (ii) that correspond to a patient's actual anatomy can be level with corresponding topographical/surface contours (e.g., at a region of interest) on the patient after registration. Additionally, or alternatively, a bulk of the reconstructed 3D volume (representing a difference between the topographical/surface contours corresponding to actual patient anatomy and topographical/surface contours of desired patient anatomy) can be external to the patient's skin after registration.

At block 228, the method 220 continues by performing navigation using the registered DICOM sequence(s). In some embodiments, the navigation can be surgical navigation. In these and other embodiments, navigation can be performed by (a) tracking a physical instrument and/or (b) displaying a representation of the physical instrument at a corresponding location within a 2D cross-sectional image and/or a reconstructed 3D volume presented on a display. For example, FIG. 9 is a partially schematic perspective view of a physical instrument 916 (e.g., a probe) contacting the forehead of a patient 990 in accordance with various embodiments of the present technology. The method 220 can track the location of the instrument 916 (e.g., relative to the patient or another marker). As the instrument 916 contacts the patient 990, a display (e.g., of a surgical navigation system, such as the display 860 of FIG. 8) can present a spatial relationship of the instrument 916 to the registered 2D cross-sectional image shown on the display and/or to a reconstructed 3D volume shown on the display. In some embodiments, presenting the spatial relationship of the instrument 916 to the registered 2D cross-sectional image and/or to the reconstructed 3D volume can include presenting a representation (e.g., the crosshairs shown in FIG. 8) of the instrument 916 at a location in the display of the 2D cross-sectional image and/or in the display of the reconstructed 3D volume. The location of the representation of the instrument 916 shown in the display can correspond to the tracked location of the instrument 916 and/or to the determined point of contact between the instrument 916 and the patient 990.

In these and other embodiments, presenting the spatial relationship can include presenting on the display a distance from (a) the tracked location of the instrument 916 and/or the determined point of contact between the instrument 916 and the patient 990 to (b) a target contour describing a desired change to patient anatomy. The distance can be displayed as a depth between the instrument 916 and the target contour. The depth can be portrayed visually as a difference between contours corresponding to actual patient anatomy and contours corresponding to desired patient anatomy at the location of the instrument 916. For example, at the location of the crosshairs in FIG. 8, there is no visible difference between the 2D slice 862 representing a contour of actual patient anatomy and the 2D slice 867 representing a desired change to the actual patient anatomy. Thus, the display 860 of FIG. 8 can indicate to a surgeon or another operator of the system that no change to the patient's actual anatomy is desired and/or needed at the location the instrument 916 (FIG. 9) is currently contacting the patient 990 (FIG. 9).

As another example, when the instrument 916 of FIG. 9 contacts the ridge or center of the nose of the patient 990, a surgical navigation system can show a representation of the instrument 916 (e.g., the crosshairs in FIG. 8) at a corresponding location on the ridge or center of the patient's nose in a display of 2D cross-sectional images and/or 3D volumes reconstructed based at least in part on the 2D cross-sectional images. Referring to FIG. 8, the surgical navigation system can further show a difference at the ridge of the patient's nose between the 2D slice 862 representing a contour of actual patient anatomy and the 2D slice 867 representing a target contour of desired patient anatomy. The difference shown in the display 860 of FIG. 8 represents a depth between the 2D slice 862 and the 2D slice 867 at least at the location the instrument 916 contacts the patient 990. This depth can provide a surgeon or another operator of the system a visual depiction of a magnitude and direction (at the location the instrument contacts that patient) that the patient's existing or real anatomy needs to be adjusted (e.g., altered, reduced, changed, etc.) to align the patient's actual anatomy with desired anatomy or a desired surgical result (represented by the 2D slice 867).

Additionally, or alternatively, the depth representing the distance between the instrument 916 and the target contour can be quantified and/or displayed. For example, if there is no difference between the patient's actual anatomy and a desired change to the patient's actual anatomy at the location the instrument 916 contacts the patient 990, the depth can be quantified and/or displayed as zero. As another example, if there is a difference between the patient's actual anatomy and a desired change to the patient's actual anatomy at the point the probe contacts the patient, the depth can be quantified and/or displayed as a positive or negative value indicating a removal or addition, respectively, (or vice versa) to the patient's actual anatomy to align the patient's actual anatomy to desired patient anatomy.

As changes to the patient's actual anatomy are made (e.g., during surgery), the depth at corresponding locations can be updated (e.g., changed, recalculated, etc.), quantified, and/or displayed (e.g., when the instrument 916 recontacts the patient 990 at those locations). Thus, a surgeon or another operator of the system is able to evaluate his/her progress towards a desired contour at a region of interest on the patient 990 simply by contacting the patient 990 with the instrument 916 at the region of interest and reviewing information presented on the display.

Although the steps of the method 220 are discussed and illustrated in a particular order, the method 220 illustrated in FIG. 2 is not so limited. In other embodiments, the method 220 can be performed in a different order. In these and other embodiments, any of the steps of the method 220 can be performed before, during, and/or after any of the other steps of the method 220. Moreover, a person of ordinary skill in the relevant art will recognize that the illustrated method 220 can be altered and still remain within these and other embodiments of the present technology. For example, one or more steps of the method 220 illustrated in FIG. 2 can be omitted and/or repeated in some embodiments.

Although not shown so as to avoid unnecessarily obscuring the description of the embodiments of the technology, any of the devices, systems, and methods described above can include and/or be performed by a computing device configured to direct and/or arrange components of the systems and/or to receive, arrange, store, analyze, and/or otherwise process data received, for example, from a 3D camera 101, a user device 105, a remote server and/or database 107, a navigation system 110, and/or other components of the system 100 of FIG. 1. As such, such a computing device includes the necessary hardware and corresponding computer-executable instructions to perform these tasks. More specifically, a computing device configured in accordance with an embodiment of the present technology can include a processor, a storage device, input/output device, one or more sensors, and/or any other suitable subsystems and/or components (e.g., displays, speakers, communication modules, etc.). The storage device can include a set of circuits or a network of storage components configured to retain information and provide access to the retained information. For example, the storage device can include volatile and/or non-volatile memory. As a more specific example, the storage device can include random access memory (RAM), magnetic disks or tapes, and/or flash memory.

The computing device can also include (e.g., non-transitory) computer readable media (e.g., the storage device, disk drives, and/or other storage media) including computer-executable instructions stored thereon that, when executed by the processor and/or computing device, cause the systems to perform one or more of the methods described herein. Moreover, the processor can be configured for performing or otherwise controlling steps, calculations, analysis, and any other functions associated with the methods described herein.

In some embodiments, the storage device can store one or more databases used to store data collected by the systems as well as data used to direct and/or adjust components of the systems. In one embodiment, for example, a database is an HTML file designed by the assignee of the present disclosure. In other embodiments, however, data is stored in other types of databases or data files.

One of ordinary skill in the art will understand that various components of the systems (e.g., the computing device) can be further divided into subcomponents, or that various components and functions of the systems may be combined and integrated. In addition, these components can communicate via wired and/or wireless communication, as well as by information contained in the storage media.

### C. EXAMPLES

Several aspects of the present technology are set forth in the following examples. Although several aspects of the present technology are set forth in examples specifically directed to methods, systems, and computer-readable mediums; any of these aspects of the present technology can similarly be set forth in examples directed to any of systems, devices, methods, and computer-readable mediums in other embodiments.
1. A method of generating a volumetric dataset for surgical navigation, the method comprising:
   obtaining a three-dimensional (3D) topographical volume of a patient representing one or more surface contours of patient anatomy;
   voxelating the 3D topographical volume to generate a 3D voxelated volume;
   volume rendering the 3D voxelated volume into a sequence of two-dimensional (2D) cross-sectional images, wherein each 2D cross-sectional image of the sequence includes a 2D slice of the 3D voxelated volume; and
   conforming the sequence of 2D cross-sectional images to an imaging standard, wherein conforming the sequence includes processing image data included in the 2D cross-sectional images of the sequence.
2. The method of example 1, further comprising:
   obtaining one or more 3D topographical images of the patient anatomy; and
   generating, based at least in part on the one or more 3D topographical images, a 3D model of the patient anatomy.
3. The method of example 2, wherein the 3D model is a composite 3D model that includes (a) a first 3D topographical volume representing actual topographical anatomy of the patient and (b) a second 3D topographical volume representing a desired change to the actual topographical anatomy of the patient.
4. The method of example 3, wherein:
   obtaining the 3D topographical volume of the patient includes obtaining the first 3D topographical volume and obtaining the second 3D topographical volume; and
   voxelating the 3D volume includes voxelating the first 3D topographical volume into a first 3D voxelated volume and voxelating the second 3D topographical volume into a second 3D voxelated volume.
5. The method of example 4, wherein volume rendering the 3D voxelated volume includes volume rendering the first 3D voxelated volume into a first sequence of 2D cross-sectional images and volume rendering the second 3D voxelated volume into a second sequence of 2D cross-sectional images.
6. The method of example 4, wherein volume rendering the 3D voxelated volume includes volume rendering the first 3D voxelated volume and the second 3D voxelated volume into a single sequence of 2D cross-sectional images.
7. The method of example 5 or example 6, wherein volume rendering includes:
   assigning pixels included in 2D slices that describe the first 3D voxelated volume, first pixel values in a first range of values that correspond to a first range of colors; and
   assigning pixels included in 2D slices that describe the second 3D voxelated volume, second pixel values in a second range of values that correspond to a second range of colors different from the first range of colors.
8. The method of any of examples 1-7, wherein:
   volume rendering the 3D voxelated volume includes advancing a frame through the 3D voxelated volume at equally spaced, non-overlapping intervals; and
   the 2D slices included in the 2D cross-sectional images of the sequence are non-overlapping slices of the 3D voxelated volume.
9. The method of any of examples 1-8, wherein voxelating the 3D topographical volume includes smoothing the 3D voxelated volume.
10. The method of any of examples 1-9, wherein the imaging standard is a Digital Imaging and Communications in Medicine (DICOM) Standard.
11. The method of example 10, wherein processing the image data includes assigning pixels of the 2D slices, new pixel values consistent with different tissue types as described in the DICOM Standard for grayscale images.
12. The method of example 10 or example 11, wherein processing the image data includes performing one or more digital measurements to determine (a) widths and heights in real word units of image frames or of the 2D slices included in the 2D cross-sectional images, and (b) one or more lengths in real word units of the 3D voxelated volume.
13. The method of any of examples 1-12, wherein conforming the sequence of 2D cross-sectional images to the imaging standard includes:
   calculating one or more dimensional attributes consistent with the imaging standard, the one or more dimensional attributes including pixel spacing, slice thickness, image position relative to the patient, or slice location;
   obtaining one or more identifiers consistent with the imaging standard, the one or more identifiers including an identifier of a method used to capture an image, an identifier of a machine used to capture the image, or an identifier of the patient;
   populating the one or more dimensional attributes or the one or more identifiers into data fields of a template compliant with the imaging standard; or
   processing the 2D cross-sectional images of the sequence such that the 2D cross-sectional images have pixel padding that is consistent with the imaging standard.
14. A method of providing surgical navigation, the method comprising:
   obtaining a volumetric dataset that includes a first sub-volume representing actual topographical anatomy of a patient and a second sub-volume representing desired topographical anatomy of the patient, wherein the desired topographical anatomy of the patient represents a desired change to the actual topographical anatomy of the patient, and wherein the volumetric dataset (a) is compliant with a Digital Imaging and Communications in Medicine (DICOM) Standard and (b) is based, at least in part, on one or more three-dimensional (3D) topographical images of the patient;
registering the volumetric dataset to the patient; and
displaying a 3D volume reconstructed based, at least in part, on two-dimensional (2D) cross-sectional images included in the volumetric dataset.
15. The method of example 14, wherein registering the volumetric dataset to the patient includes registering the first sub-volume to existing topographical anatomy of the patient.
16. The method of example 15, wherein registering the volumetric dataset to the patient further includes overlaying the second sub-volume onto the first sub-volume using a best fit algorithm.
17. The method of any of examples 14-16, wherein registering the volumetric dataset to the patient includes registering the volumetric dataset to the patient such that a bulk of the volumetric dataset is positioned internal to the patient.
18. The method of any of examples 14-17, wherein displaying the 3D volume includes displaying a difference between the first sub-volume and the second sub-volume, and wherein the difference represents a depth between the patient's actual topographical anatomy and the patient's desired topographical anatomy.
19. The method of any of examples 14-18, wherein the displaying the 3D volume includes:
   tracking a position of a physical instrument; and
   displaying a difference between the first sub-volume and the second sub-volume at a location that the physical instrument contacts the patient.
20. A modeling and navigation system, comprising:
   a three-dimensional (3D) imaging device configured to obtain one or more 3D topographical images of patient anatomy;
   a computing device configured to
      generate one or more 3D models based, at least in part, on the one or more 3D topographical images, wherein the one or more 3D models include a representation of actual topographical patient anatomy and a representation of desired topographical patient anatomy,
      voxelate the one or more 3D models into one or more 3D voxelated volumes,
      volume render the one or more 3D voxelated volumes into one or more sequences of 2D cross-sectional images,
      process image data included in the 2D cross-sectional images, and conform the one or more sequences of 2D cross-sectional images to an imaging standard; and
   a surgical navigation system configured to
      reconstruct a 3D volume based, at least in part, on the one or more sequences of 2D cross-sectional images that conform to the imaging standard,
      register the 3D volume to the patient,
   track a position of a physical instrument, and
   display the 3D volume and a difference between the actual topographical patient anatomy and the desired topographical patient anatomy at a location that the physical instrument contacts the patient.
21. A non-transitory, computer-readable medium having instructions stored thereon that, when executed by one or more processors of a modeling system, cause the modeling system to perform a method comprising:
   obtaining a three-dimensional (3D) topographical volume of a patient representing one or more surface contours of patient anatomy;
   voxelating the 3D topographical volume to generate a 3D voxelated volume;
   volume rendering the 3D voxelated volume into a sequence of two-dimensional (2D) cross-sectional images, wherein each 2D cross-sectional image of the sequence includes a 2D slice of the 3D voxelated volume; and
   conforming the sequence of 2D cross-sectional images to an imaging standard, wherein conforming the sequence includes processing image data included in the 2D cross-sectional images of the sequence.
22. A non-transitory, computer-readable medium having instructions stored thereon that, when executed by one or more processors of a navigation system, cause the navigation system to perform a method comprising:
   obtaining a volumetric dataset that includes a first sub-volume representing actual topographical anatomy of a patient and a second sub-volume representing desired topographical anatomy of the patient, wherein the desired topographical anatomy of the patient represents a desired change to the actual topographical anatomy of the patient, and wherein the volumetric dataset (a) is compliant with an imaging standard and (b) is based, at least in part, on one or more three-dimensional (3D) topographical images of the patient;
   registering the volumetric dataset to the patient; and
   displaying a 3D volume reconstructed based, at least in part, on two-dimensional (2D) cross-sectional images included in the volumetric dataset.

### D. Conclusion

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context permits, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. Moreover, the terms “connect” and “couple” are used interchangeably herein and refer to both direct and indirect connections or couplings. For example, where the context permits, element A “connected” or “coupled” to element B can refer (i) to A directly “connected” or directly “coupled” to B and/or (ii) to A indirectly “connected”or indirectly “coupled”to B.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. As another example, various components of the technology can be further divided into subcomponents, and/or various components and/or functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology.

It should also be noted that other embodiments in addition to those disclosed herein are within the scope of the present technology. For example, embodiments of the present technology can have different configurations, components, and/or procedures in addition to those shown or described herein. Moreover, a person of ordinary skill in the art will understand that these and other embodiments can be without several of the configurations, components, and/or procedures shown or described herein without deviating from the present technology. Accordingly, the disclosure and associated technology can encompass other embodiments not expressly shown or described herein.

1. A method of generating a volumetric dataset for surgical navigation, the method comprising:
obtaining a three-dimensional (3D) topographical volume of a patient representing one or more surface contours of patient anatomy;
voxelating the 3D topographical volume to generate a 3D voxelated volume;
volume rendering the 3D voxelated volume into a sequence of two-dimensional (2D) cross-sectional images, wherein each 2D cross-sectional image of the sequence includes a 2D slice of the 3D voxelated volume; and
conforming the sequence of 2D cross-sectional images to an imaging standard, wherein conforming the sequence includes processing image data included in the 2D cross-sectional images of the sequence.

2. The method of claim 1, further comprising:
obtaining one or more 3D topographical images of the patient anatomy; and
generating, based at least in part on the one or more 3D topographical images, a 3D model of the patient anatomy.

3. The method of claim 2, wherein the 3D model is a composite 3D model that includes (a) a first 3D topographical volume representing actual topographical anatomy of the patient and (b) a second 3D topographical volume representing a desired change to the actual topographical anatomy of the patient.

4. The method of claim 3, wherein:
obtaining the 3D topographical volume of the patient includes obtaining the first 3D topographical volume and obtaining the second 3D topographical volume; and
voxelating the 3D topographical volume includes voxelating the first 3D topographical volume into a first 3D voxelated volume and voxelating the second 3D topographical volume into a second 3D voxelated volume.

5. The method of claim 4, wherein volume rendering the 3D voxelated volume includes volume rendering the first 3D voxelated volume into a first sequence of 2D cross-sectional images and volume rendering the second 3D voxelated volume into a second sequence of 2D cross-sectional images.

6. The method of claim 4, wherein volume rendering the 3D voxelated volume includes volume rendering the first 3D voxelated volume and the second 3D voxelated volume into a single sequence of 2D cross-sectional images.

7. The method of claim 5, wherein volume rendering includes:
assigning pixels included in 2D slices that describe the first 3D voxelated volume, first pixel values in a first range of values that correspond to a first range of colors; and
assigning pixels included in 2D slices that describe the second 3D voxelated volume, second pixel values in a second range of values that correspond to a second range of colors different from the first range of colors.

8. The method of claim 1, wherein:
volume rendering the 3D voxelated volume includes advancing a frame through the 3D voxelated volume at equally spaced, non-overlapping intervals; and
the 2D slices included in the 2D cross-sectional images of the sequence are non-overlapping slices of the 3D voxelated volume.

9. The method of claim 1, wherein voxelating the 3D topographical volume includes smoothing the 3D voxelated volume.

10. The method of claim 1, wherein the imaging standard is a Digital Imaging and Communications in Medicine (DICOM) Standard.

11. The method of claim 10, wherein processing the image data includes assigning, consistent with different tissue types as described in the DICOM Standard for grayscale images, new pixel values to pixels of the 2D slices.

12. The method of claim 10, wherein processing the image data includes performing one or more digital measurements to determine (a) widths and heights in real word units of image frames or of the 2D slices included in the 2D cross-sectional images, and (b) one or more lengths in real word units of the 3D voxelated volume.

13. The method of claim 1, wherein conforming the sequence of 2D cross-sectional images to the imaging standard includes:
calculating one or more dimensional attributes consistent with the imaging standard, the one or more dimensional attributes including pixel spacing, slice thickness, image position relative to the patient, or slice location;
obtaining one or more identifiers consistent with the imaging standard, the one or more identifiers including an identifier of a method used to capture an image, an identifier of a machine used to capture the image, or an identifier of the patient;
populating the one or more dimensional attributes or the one or more identifiers into data fields of a template compliant with the imaging standard; or
processing the 2D cross-sectional images of the sequence such that the 2D cross-sectional images have pixel padding that is consistent with the imaging standard.

14. A method of providing surgical navigation, the method comprising:
obtaining a volumetric dataset that includes a first sub-volume representing actual topographical anatomy of a patient and a second sub-volume representing desired topographical anatomy of the patient, wherein the desired topographical anatomy of the patient represents a desired change to the actual topographical anatomy of the patient, and wherein the volumetric dataset (a) is compliant with a Digital Imaging and Communications in Medicine (DICOM) Standard and (b) is based, at least in part, on one or more three-dimensional (3D) topographical images of the patient;
registering the volumetric dataset to the patient; and
displaying a 3D volume reconstructed based, at least in part, on two-dimensional (2D) cross-sectional images included in the volumetric dataset.

15. The method of claim 14, wherein registering the volumetric dataset to the patient includes registering the first sub-volume to existing topographical anatomy of the patient.

16. The method of claim 15, wherein registering the volumetric dataset to the patient further includes overlaying the second sub-volume onto the first sub-volume using a best fit algorithm.

17. The method of claim 14, wherein registering the volumetric dataset to the patient includes registering the volumetric dataset to the patient such that a bulk of the volumetric dataset is positioned internal to the patient.

18. The method of claim 14, wherein displaying the 3D volume includes displaying a difference between the first sub-volume and the second sub-volume, and wherein the difference represents a depth between the patient's actual topographical anatomy and the patient's desired topographical anatomy.

19. The method of claim 14, wherein the displaying the 3D volume includes:
tracking a position of a physical instrument; and
displaying a difference between the first sub-volume and the second sub-volume at a location that the physical instrument contacts the patient.

20. A modeling and navigation system, comprising:
a three-dimensional (3D) imaging device configured to obtain one or more 3D topographical images of patient anatomy;
a computing device configured to
generate one or more 3D models based, at least in part, on the one or more 3D topographical images, wherein the one or more 3D models include a representation of actual topographical patient anatomy and a representation of desired topographical patient anatomy,
voxelate the one or more 3D models into one or more 3D voxelated volumes,
volume render the one or more 3D voxelated volumes into one or more sequences of 2D cross-sectional images,
process image data included in the 2D cross-sectional images, and
conform the one or more sequences of 2D cross-sectional images to an imaging standard; and
a surgical navigation system configured to
reconstruct a 3D volume based, at least in part, on the one or more sequences of 2D cross-sectional images that conform to the imaging standard,
register the 3D volume to the patient,
track a position of a physical instrument, and
display the 3D volume and a difference between the actual topographical patient anatomy and the desired topographical patient anatomy at a location that the physical instrument contacts the patient.

21. (canceled)

22. (canceled)
